(19) United States
(12) Patent Application Publication
(10) Pub. No.: US 2025/0064407 A1
(43) Pub. Date: Feb. 27, 2025

(54) TELEMEDICINE SUPPORT SYSTEM FOR ASSISTING SELECTION AND EXECUTION OF REHABILITATION PROGRAM FOR ORTHOPEDIC PATIENT

(71) Applicant: UT HEALTHTECH CO., LTD., Fukushima (JP)

(72) Inventors: Yuya Kurihara, Fukushima (JP); Osamu Shirato, Hokkaido (JP); Tatsuya Endo, Fukushima (JP); Kazuya Oribe, Fukushima (JP)

(73) Assignee: UT HEALTHTECH CO., LTD., Fukushima (JP)

(21) Appl. No.: 18/912,113

(22) Filed: Oct. 10, 2024

Related U.S. Application Data
(63) Continuation-in-part of application No. PCT/JP2023/017282, filed on May 8, 2023.

(30) Foreign Application Priority Data
May 9, 2022 (JP) 2022-077164

(51) Int. Cl. A61B 5/00; A61B 5/0205; A61B 5/024; G16H 10/60; G16H 20/30; G16H 80/00

(52) U.S. Cl. CPC A61B 5/7465; A61B 5/0004; A61B 5/0022; A61B 5/0205; A61B 5/4866; A61B 5/6802; G16H 10/60; G16H 20/30; G16H 80/00; A61B 5/024

(57) ABSTRACT

Telemedicine support system for assisting a selection and execution of a rehabilitation program for an orthopedic patient is disclosed. The system assists selection of a rehabilitation program suitable for the patient in accordance with rehabilitation index that is calculated based on the evaluation score obtained by using an interview sheet of medical questionnaire. The system updates the rehabilitation program to a program grade having more suitable load for the patient based on the comparison between exercise biometric data of the patient and exercise target value of the patient for the rehabilitation program.

### TECHNICAL FIELD

This invention relates to a telemedicine support system using a mobile phone that assists selecting and executing a rehabilitation program for an orthopedic patient who is capable of performing an independent exercise.

### BACKGROUND ART

Since orthopedic patients having a locomotor disease or disorder have reduced exercise capability after completion of a treatment of their disease, a period of rehabilitation is necessary for those patients to recover health condition. Rehabilitation program is prescribed for patients to recover their motor function after the treatment of disease.

A rehabilitation program for orthopedic patients who are capable of performing an independent exercise is prescribed by a physician after examination of the patient. The physician provides an exercise instruction that is tailored to the patient's profile and condition confirmed by an examination. A rehabilitation usually takes several months or longer, depending on the kind and extent of the disease. And the patient's condition changes during the recovery process. Therefore, during the rehabilitation period, the patient regularly needs to go to hospital to check his or her health status from time to time and update the rehabilitation program so that the load of the rehabilitation program is appropriate for the patient. As a result, patients are forced to go to hospital regularly for a long period of time, and hospitals are always crowded with outpatients.

Recently, a telemedicine system using a computer network has been proposed for providing rehabilitation instruction to patients. By sending a video to a patient via a computer network, the patient can easily and effectively receive rehabilitation instruction without going to hospital.

In a telemedicine system, since the physician does not have a face-to-face interview nor palpate the patient, an interview sheet is used to examine the patient. The interview sheet asks a number of questions, and the patient's health condition is ascertained based on the patient's answers to the questions. Currently used interview sheet consists of questions that are developed according to a health-related quality of life scale (e.g., SF-36) for the patient's health status and questions that are developed according to a disease-specific scale (e.g., RDQ) related to the disease of the patient. These two scales are often used in combination in medical settings to assess a patient's disease recovery status.

In order to prescribe a rehabilitation program in accordance with the progress of recovery of the patient, result of evaluation by interview sheet needs to be provided in a form of quantitative data. SF-36 is a scientific, reliable, and valid measurement scale for quantitative assessment of health-related quality of life (HRQOL). Roland Morris Disability Questionnaire (RDQ) is a measurement scale that allows a patient to quantitatively assess his/her own disability from daily living due to low back pain. These instruments consist of a number of questions, and based on the answers to the questions, the patient's recovery status is assessed by the sum of the scores assigned to the answer for each question. Quantitative assessments of patients' disease recovery status based on these instruments are internationally approved and reliable.

However, a medical interview sheet, no matter how well it is prepared, relies on the patient's self-report. Also, in order for the result of the interview to be accurate, the patient must be able to understand the questions accurately and be aware of his or her own health condition with respect to the questions asked. If the patient is an elderly person suffering from dementia, it is difficult to ask for answers to a large number of questions. Therefore, use of a questionnaire, while useful, has limitation. by itself. In addition, the patient's health status changes with the progress of rehabilitation. In order to ensure that load of the initially appropriately selected rehabilitation program is compatible with the patient's current health status, it is necessary to update the assessment of the patient's health status and update the rehabilitation program prescribed for the patient in accordance with the updated assessment.

### Measure to Solve the Problem

Under the circumstances described above, inventors of the present invention have made an intensive study to solve above problems, and as a result, have developed a telemedicine support system that assists selecting and executing a rehabilitation program for an orthopedic patient who is capable of performing an independent exercise,
the system comprises a mobile electronic device and a PC (personal computer) that is wirelessly connected to the mobile electronic device,
the mobile electronic device is configured to be carried by a patient, and comprises:
   means for accepting an input of identification number, profile, and a kind of disease of the patient,
   means for accepting an input of a health evaluation score that quantitatively assesses the patient's health status,
   means for measuring and collecting exercise biometric data of the patient, and
   means for transmitting the data of the patient to the PC,
the PC is installed in a medical institution and accessible by a physician of the patient, and comprises:
   means for receiving data of the patient transmitted from the mobile electronic device over an Internet line, and
   a memory device,
      the memory device stores a plurality of rehabilitation programs, each of the plurality of rehabilitation programs consists of a plurality of grades in accordance with magnitude of load that is applied to the patient by performing the exercise of the program, each grade of the rehabilitation program is provided with a rehabilitation index of the patient having a health status suitable for the load of the exercise program of the grade,
      the memory device further stores a target value of exercise biometric data (exercise target value) for each grade of the rehabilitation program and a profile of the patient who is expected to perform the exercise of the rehabilitation program with the exercise target value,
upon receiving data of the patient from the mobile electronic device, the PC
   determines the rehabilitation index that assesses health status of the patient by a numerical scale based on the health evaluation score of the patient,
   select a rehabilitation program and a grade of the rehabilitation program to be prescribed for the patient from a plurality of rehabilitation programs stored in the memory based on the kind of disease and the rehabilitation index of the patient, and
   determines an exercise target value of the patient for the selected grade of the rehabilitation program based on the profile of the patient,
the system compares a value of exercise biometric data measured from the patient who performed the selected grade of rehabilitation program with the exercise target value of the patient for the selected grade of rehabilitation program,
when the value of the exercise biometric data measured by the mobile electronic device is within a predetermined upper and lower range from the exercise target value in accordance with a predetermined rule, propose using the prescribed rehabilitation program based on a judgement that the rehabilitation program that was prescribed for the patient was not overload nor underload for the patient,
when the value of the exercise biometric data measured by the mobile electronic device is beyond a predetermined upper range from the exercise target value in accordance with the predetermined rule, propose changing the prescribed rehabilitation program into a grade of the rehabilitation program having one level lower load based on a judgement that the rehabilitation program that was prescribed for the patient was overload for the patient, and
when the value of the exercise biometric data measured by the mobile electronic device is below a predetermined lower range from the exercise target value in accordance with the predetermined rule, propose changing the prescribed rehabilitation program into a grade of the rehabilitation program having one level higher load based on a judgement that the rehabilitation program that was prescribed for the patient was underload for the patient.

Preferably, exercise biometric data of the patient is measured over a plurality of measurement items, and exercise biometric data of the patient is compared with the exercise target value of the patient for each measurement item. Large or small points are awarded to the result of each comparison depending on the deviation from the exercise target value of the patient, and the awarded points are summed up over the plurality of measurement items to produce a total value. If the total value is beyond or below a predetermined threshold value, the load of the selected grade of rehabilitation program is determined to have been overload or underload for the patient, and a rehabilitation program of the grade of one level lower or higher load than the prescribed rehabilitation program is proposed.

Preferably, if any one of the exercise biometric data of patient measured over the plurality of measurement items by the mobile electronic device is above or below a predetermined upper or lower range from the exercise target value of the patient, the load of the selected grade of rehabilitation program is determined to have been overload or underload for the patient, and a rehabilitation program of the grade of one level lower or higher load than the prescribed rehabilitation program is proposed.

Preferably, exercise biometric data of the patient are calorie consumption and heart rate.

Preferably, calorie consumption by the patient is calculated based on the exercise intensity (METs value) of independent exercise, exercise time of the exercise program, and weight of the patient.

Preferably, profile of the patient includes all or part of the patient's age, gender, height, and weight.

Preferably, exercise biometric data of the patient includes all or part of calories consumed, heart rate, walking distance, walking speed, and number of steps of the patient.

Preferably, the mobile electronic device includes a mobile phone and a wearable sensor device that is worn on a body of the patient and connected wirelessly or wired to the mobile phone so that data of heart rate of the patient can be measured by the wearable sensor device and transmitted to the mobile phone.

Preferably, the means of measuring and collecting exercise biometric data of the patient of the mobile electronic device includes Google Fit (registered trademark).

### Advantage of the Invention

In an embodiment of the system of present invention, since a commercially available mobile phone having an application software for measuring exercise biometric data installed can be used as the mobile electronic device, the system is convenient, high performance, and low cost for the user, making the system a commercially practical choice.

In an embodiment of the system of present invention, the predetermined upper and lower range from the exercise target values is set taking into account of clinical consideration of a doctor so that the decision of updating the rehabilitation program is made reliable and safe.

In an embodiment of the system of present invention, exercise target value for a grade of rehabilitation program is set in combination with a profile of a patient who performs the grade of rehabilitation program. Therefore, prescribed rehabilitation program can be updated taking into account of the characteristic of each patient.

In an embodiment of the system of present invention, because a grade of rehabilitation program is updated based on measurement data of a plurality of measurement items, the system is more reliable than a system that updates the grade of rehabilitation program based on a measurement data of one measurement item.

In an embodiment of the system of the present invention, exercise intensity (METs value) of the exercise program is registered in the rehabilitation program, and calorie consumption is calculated based on the METs value, exercise time and patient profile (body weight). Therefore, the system can be applied to all rehabilitation programs regardless of the content of exercise.

### BRIEF DESCRIPTION OF DRAWINGS

FIG. 1 shows a conceptual diagram of the network of the system of the present invention.

FIG. 2 shows a functional configuration diagram of a PC used in the system of the present invention.

FIG. 3 shows a functional diagram of a mobile electronic device used in the system of the present invention.

FIG. 4 shows an example of the display screen of a mobile electronic device used in the system of the present invention.

FIG. 5 shows an example of SF-36 Questionnaire that is used in the system of the present invention.

FIG. 6 shows an example of an RMDQ questionnaire that is used in the system of the present invention.

FIG. 7 shows an example of the questions in the medical questionnaire used in the system of the present invention.

FIG. 8 shows the relationship between health evaluation score, rehabilitation index, and rehabilitation program (for patients with low back pain) used in the system of the present invention.

FIGS. 9(A) and 9(B) show examples of setting target heart rate and target calorie consumption (for patients with low back pain) of a patient who performed one unit of rehabilitation program (15 minutes exercise) in an embodiment of the present invention. FIG. 9(A) shows an example of male patients having different age, height, and weight. FIG. 9(B) shows an example of female patients having different age, height, and weight.

FIG. 10 shows examples of selecting and updating a rehabilitation program (for low back pain patients) in an embodiment of the present invention.

FIG. 11 shows the relationship between grades of a rehabilitation program and rehabilitation index of a patient for various kinds of diseases of a patient in an embodiment of the invention.

FIG. 12 shows the method of determining exercise target value of a patient for each measurement item.

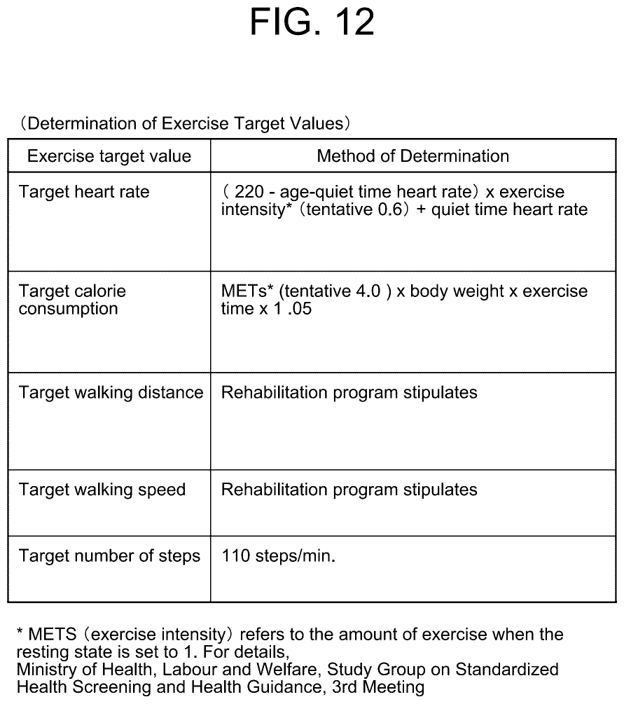

FIG. 13: shows a method of determining overload/underload of an exercise program for a rehabilitation program in an embodiment of the present invention.

### DETAILED DESCRIPTION OF THE INVENTION

### Definition.

### <Independent Exercise>

In the present invention, “independent exercise” refers to an exercise that a patient can perform by himself/herself under an instruction from a physician or physical therapist, which may include muscle strengthening exercise, muscle endurance building exercise, cardiopulmonary capacity building exercise (whole body endurance building exercise) flexibility improving exercise, skillfulness improving exercise, coordination improving exercise and running ability improving exercise. Walking exercise falls under muscle strengthening, muscle endurance building, and cardiopulmonary capacity building exercise. Exercise of standing up from a chair falls under muscle strengthening and muscle endurance building exercises.

### <Health-Related Quality of Life Rating Scale>

In the present invention, “health-related QOL” refers to the quantified impact of a disease or treatment on a patient's subjective sense of health (mental health, vitality, pain, etc.) and work, household, and social activities performed daily. A “health-related quality of life rating scale” refers to a scale used to objectively assess health-related quality of life, which may use a disease-nonspecific scale (e.g. SF-36) and a disease-specific scale (e.g. RDQ).

### <Health Evaluation Score>

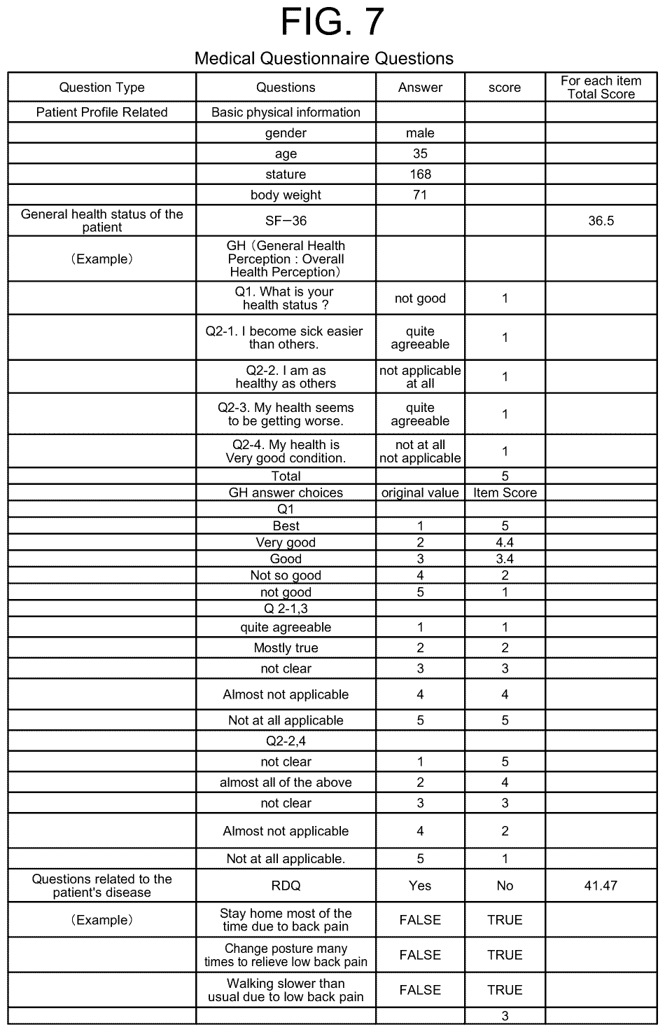

In the present invention, “health evaluation score” refers to a score calculated based on patient's responses to each question in a medical questionnaire. Recovery status of a patient is assessed by a sum of the scores assigned to the answer (e.g. yes or no) to each question of the questionnaire. Health evaluation score is used as a basis for calculating the rehabilitation index. In a preferred embodiment of the invention, the medical questionnaire includes non-disease-specific and/or disease-specific questionnaire items, and health status of a patient is evaluated in the form of a quantitative score based on the responses by the patient to these questionnaire items. However, in a case where it is difficult to obtain answers to a large number of questions from the patient, physician or physiotherapist can determine the score, taking into account the distribution of scores in the medical questionnaire. In addition, the physician or physical therapist can add a numerical value based on the patient's profile (gender, age, height, weight, etc.) to the health evaluation score (see FIG. 7).

### <Disease-Nonspecific Score>

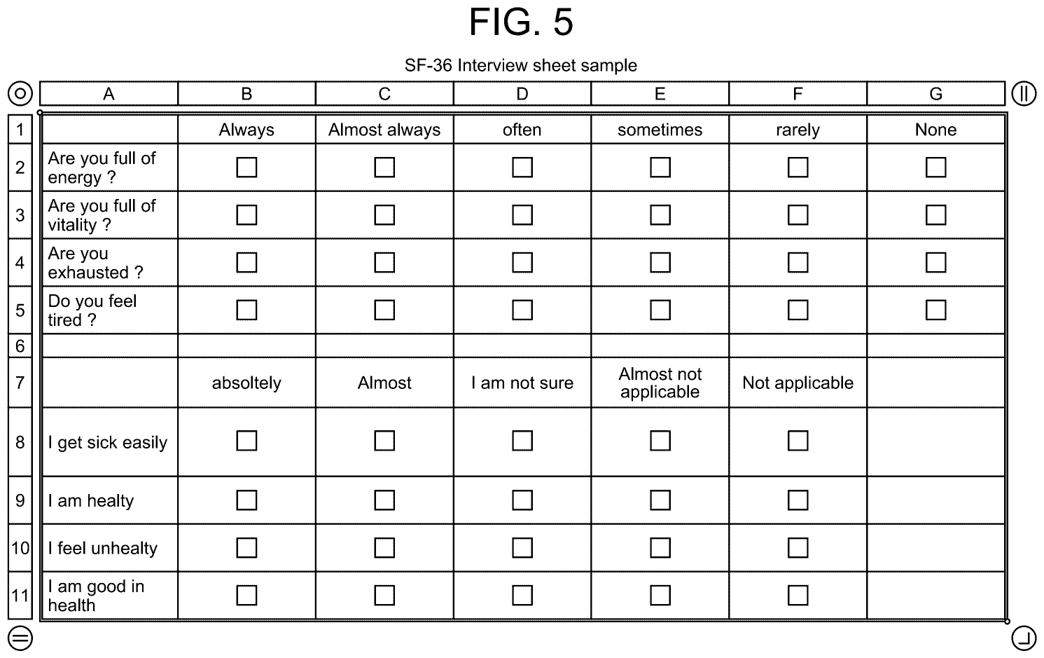

In the present invention, “disease-nonspecific score” is a score obtained from an interview based on health-related quality of life rating scale, regardless of a specific disease which the patient may have. SF-36 is used as a representative disease-nonspecific score (See FIG. 5).

### <Disease-Specific Score>

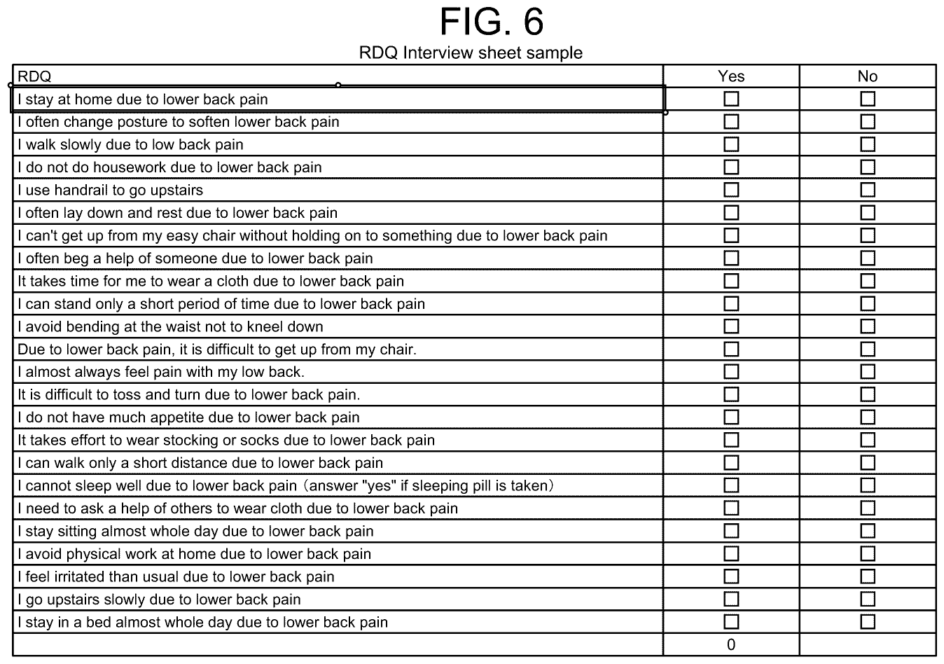

In the present invention, “disease-specific score” is a score calculated in connection with the specific disease of the patient. It is calculated based on the score obtained by interviewing the patient using a questionnaire of a disease-specific quality of life scale. The RDQ (Roland Morris Disability Questionnaire) is a scale that allows patients to quantitatively assess their own disability in daily living due to low back pain (see FIG. 6).

### <Rehabilitation Index>

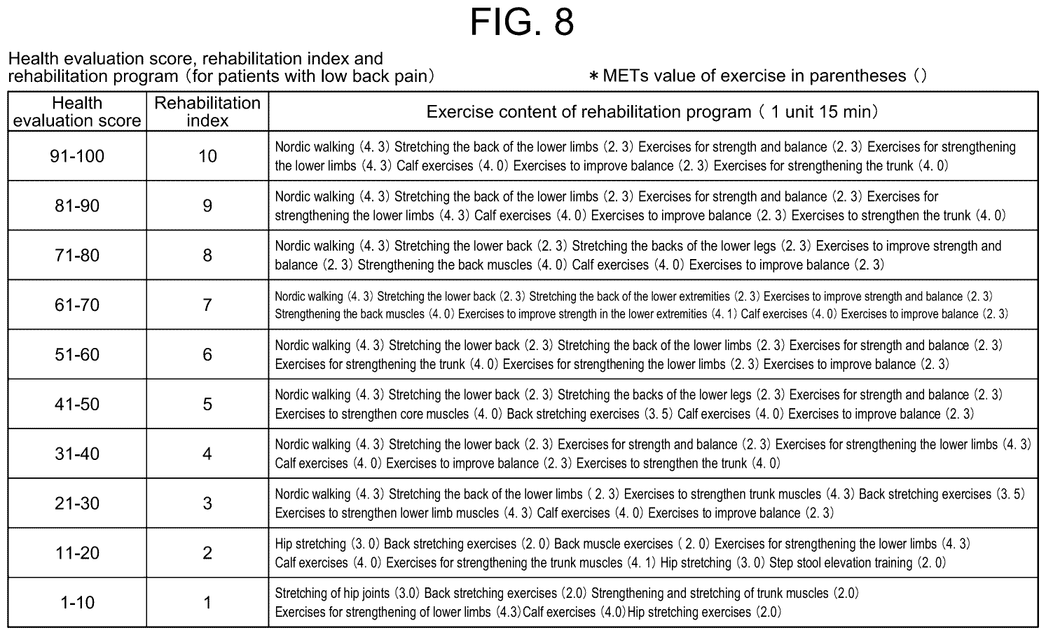

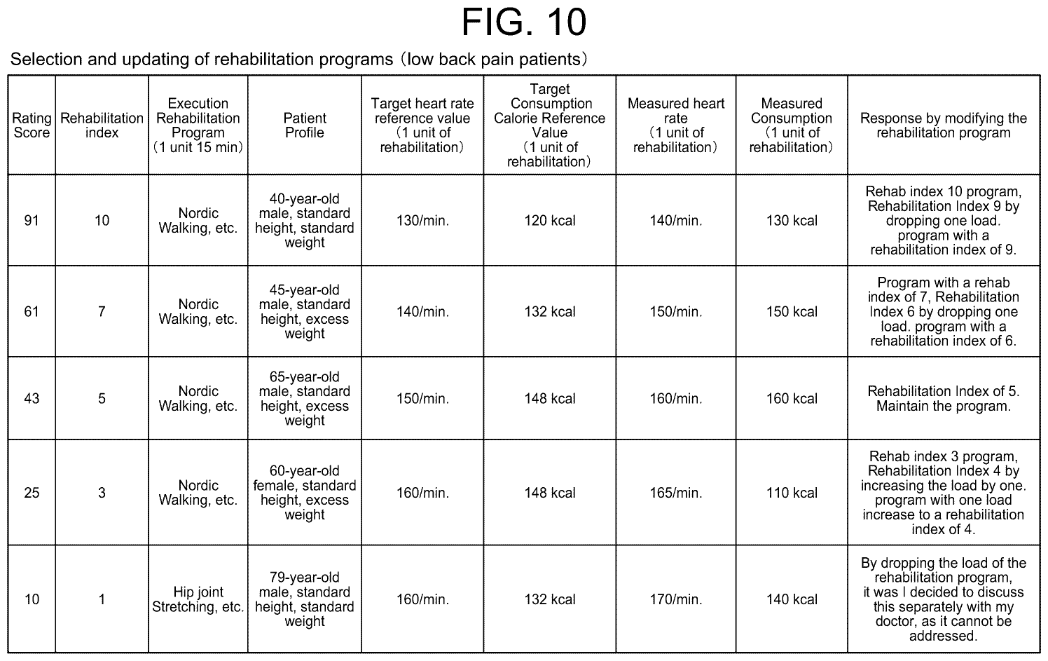

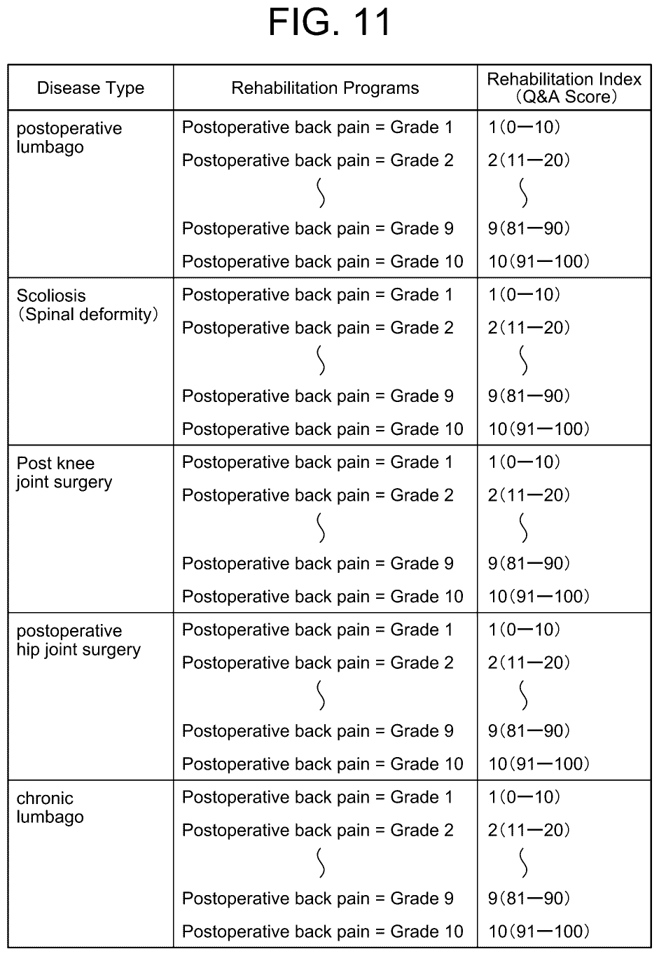

In the present invention, “rehabilitation index” refers to a value of health status of a patient rated on a numerical scale. Rehabilitation Index is calculated based on the health evaluation score. In a preferred embodiment of the invention, when the SF-36 is used as a medical questionnaire, the SF medical questionnaire evaluation score 1 to 100 is divided into ten levels such that 1 to 10 is rehabilitation index 1, 11 to 20 is rehabilitation index 2, 21 to 30 is rehabilitation index 3, 31 to 40 is rehabilitation index 4, 41 to 50 is rehabilitation index 5, 51 to 60 is rehab index 6, 61 to 70 is rehab index 7, 71 to 80 is rehab index 8, 81 to 90 is rehab index 9, and 91 to 100 is rehab index 10 (see FIGS. 8 and 11). If the total score resulting from the SF-36 interview of a patient is 43, the rehabilitation index for that patient is 5. If a disease-nonspecific score (SF-36) and a disease-specific score (RMDQ) are used for the interview, the rehabilitation index for the patient would be 3 if the average of the sum of the SF-36 and RMDQ assessment scores divided by 2 is 25 (see FIG. 8 and FIG. 10).

In addition to disease-specific factors in which cause of disease is clear, disease-nonspecific factors in which cause of the disease is unclear often play a major role to cause a disease such as low back pain. In selecting a rehabilitation program for a patient having low back pain, RDQ is used as the evaluation criterion related to the patient's back disease. However, low back pain may be due to factors other than back disease, which should be evaluated using a disease-nonspecific scale.

In the present invention, disease of an orthopedic patient is interviewed by both the questionnaire items based on the disease-specific scale and the questionnaire items based on the disease-nonspecific scale. Rehabilitation index is calculated based on the results of the interview by both of these two questionnaire items. Based on the rehabilitation index thus calculated, a rehabilitation program and the grade of the program for a patient having a specific disease is selected.

In addition to the health evaluation score obtained from interview using SF-36, RMDQ, etc., a physician or physical therapist may determine the rehabilitation index taking into account of other characteristic or profile of the patient that is not assessed in such an interview. For example, if the patient is older and heavier for his/her height, the rehabilitation program to be prescribed should be less strenuous, so the rehabilitation index can be set smaller than that calculated from the medical interview score.

### <Mobile Electronic Device>

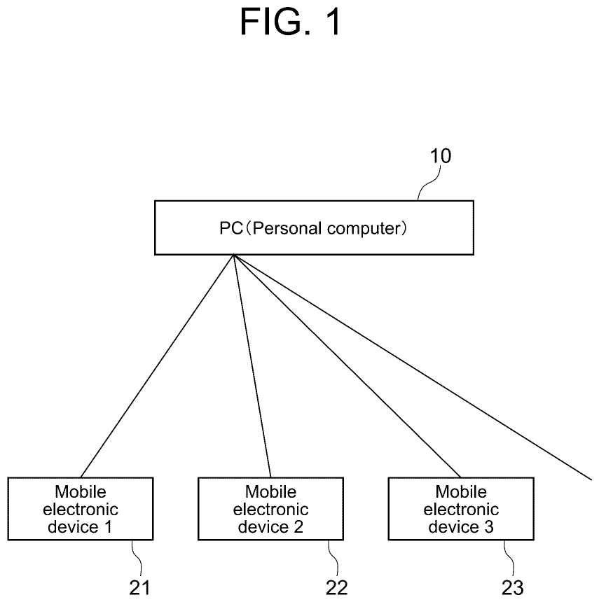

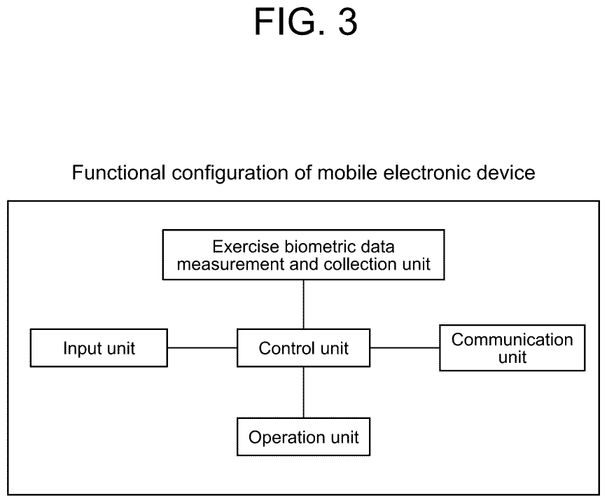

In the present invention, “mobile electronic device” refers to an electronic device that can be carried by the patient to measure and collect exercise biometric data of the patient. Mobile electronic device is provided with the functionality to perform the system of the invention (see FIG. 3). The mobile electronic device is wirelessly connected to a PC that is installed in a medical institution (see FIG. 1). The mobile electronic device may include a mobile phone. The mobile phone can be provided with application software for measuring exercise biometric data of a patient (e.g. Google Fit™). The patient's heart rate may not be measured by the mobile phone. However, patient's heart rate can be measured by a sensor device that is wrapped around the patient's body or arm and connected to the mobile phone by wire or wirelessly,

### <PC (Personal Computer)>

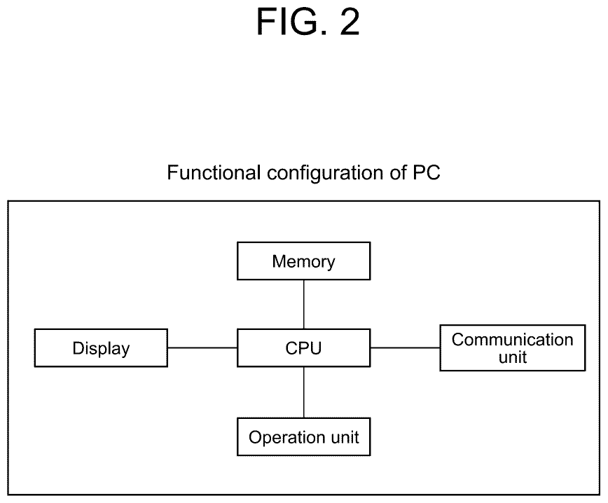

In the present invention, “PC (personal computer)” refers to a device connected to a mobile electronic device and having the capability to execute the system of the invention (see FIG. 2). The PC is connected wirelessly with the mobile electronic device (see FIG. 1).

### <Exercise Biometric Data>

In the present invention, “exercise biometric data” refers to data measured by a mobile electronic device from a patient who performed an exercise of a rehabilitation program. Exercise biometric data may include data of calorie consumption, heart rate, walking distance (in one session of program), walking speed, or number of walking steps of a patient who performed an exercise program prescribed for the patient.

### <Exercise Target Value>.

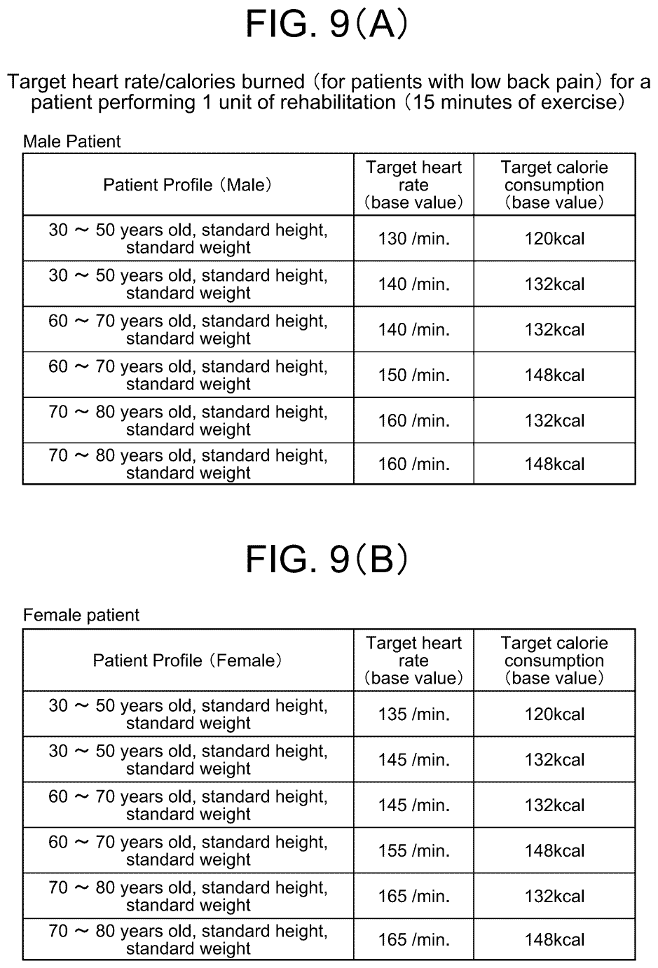

In the present invention, “exercise target value” refers to a target value that is expected to be measured from the patient performing the exercise of the rehabilitation program. Exercise target value is stored in the memory device of PC for each grade of rehabilitation program together with a profile of the patient who performs the exercise of the program (FIG. 9, 10).

In an embodiment of the invention, a predetermined upper and lower range from the exercise target value is set as a threshold value. The predetermined upper and lower range is used to compare the value of biometric data of the patient with the exercise target value. If the value of biometric data measured from the patient who performed the exercise program is within the predetermined upper/lower range from the exercise target value, the load of the exercise program is determined to be appropriate (not overload nor underload). In a preferred embodiment of the invention, the predetermined range is set ±10% from the exercise target value. Clinical physician or physical therapist can decide what percentage of the range above or below the exercise target value to set.

Target value of an exercise (target heart rate, target calorie consumption, target walking distance, target number of steps, and target walking speed) are not the same for all patients who perform the same exercise. Even if the same exercise is performed, their exercise biometric data would be different depending on a profile of individual patient (age, gender, weight, and height). In an embodiment of present invention, correspondence between an exercise of rehabilitation program, patient profile, and exercise target value are stored in the memory device of PC and used to calculate the exercise target values for individual patients who perform the exercise of the rehabilitation program. FIG. 9 shows an example of the correspondence between a profile of patient, target heart rate, and target calorie consumption in a rehabilitation program for patients having low back pain.

### <Target Calorie Consumption>

In the present invention, “target calorie consumption” refers to the calories that are expected to be consumed by a patient who performs the exercise of the rehabilitation program. In an embodiment of the invention, +10% is set as the predetermined upper and lower range from the target calorie consumption (exercise target value) that is compared with the value of biometric data the patient.

Generally, amount of energy consumed in one exercise session (e.g. 15 minutes) is determined by the patient's body size (weight), exercise intensity and exercise duration. Since the amount of energy (calories) that a patient consumes by performing the exercise depends on the weight and other factors of each individual patient, target calorie consumption is set depending on a profile of the patient.

In an embodiment of the present invention, if the calorie consumption measured by the mobile electronic device of a patient who performed the exercise of the prescribed rehabilitation program exceeds the upper limit of the target calorie consumption range, the exercise is determined to be overload for the patient.

### <METs Value>.

MET stands for Metabolic Equivalent of Task. One MET is the amount of energy used while sitting quietly. Physical activities may be rated using METs to indicate their intensity. For example, reading may use about 1.3 METs while running may use 8-9 METs. The exercises in the rehabilitation program may include Nordic walking, stretching the back of the lower limbs, exercises for strength and balance, exercises for strengthening the muscles of the lower limbs, calf exercises, balance improvement exercise, exercises for strengthening core muscles, and strengthening muscles of the body. Since each of these exercises has different intensity of exercise, these exercises have different METs values. The system of the present invention allows patients to calculate the calorie consumption that is associated with performing the exercise of prescribed rehabilitation program by registering the METs values of the exercises in each rehabilitation program stored in the PC.

### <Target Heart Rate>

In the present invention, “target heart rate” is a target value of heart rate that is expected to be measured from a patient after the patient performed the exercise of the rehabilitation program. Generally, patient's heart rate increases with a higher load applied by the exercise and decreases with a lower load. Since the load applied to a patient by performing the indicated exercise varies depending on the gender, age, height, weight, etc. of each patient, the target heart rate is set according to the gender, age, height, weight, etc. of the patient. In an embodiment of this invention, if the value of heart rate measured and collected by the mobile electronic device from a patient who performed the walking exercise instructed by the prescribed rehabilitation program exceeds the value of the predetermined upper range from the target heart rate, the walking exercise is determined to be overload for the patient.

### <Target Walking Distance>

In the present invention, “target walking distance” refers to the distance that a patient is expected to walk within a predetermined time after performing the walking exercise that is instructed by the rehabilitation program. The target walking distance is set in each rehabilitation program stored in the memory in a manner corresponding to profile of the patient. In the present invention, if the walking distance measured and collected by a mobile electronic device is less than the target walking distance, the exercise is determined to be overload for the patient.

### <Target Walking Speed>

In the present invention, “target walking speed” refers to the speed of walking by a patient that is expected to be measured from the patient who performed the walking exercise that is instructed in the rehabilitation program. The target walking speed is set in each rehabilitation program stored in the memory corresponding to the patient profile. If the measured walking speed collected by the mobile electronic device is below the predetermined lower range from the target walking speed, the exercise is determined to be overload for the patient.

### EMBODIMENTS

FIG. 1 shows the network configuration of an embodiment of the system of present invention. A personal computer (PC) of a physician in charge of the patient is connected wirelessly to mobile electronic devices 1, 2, and 3 that are carried by patients 1, 2, and 3, respectively.

### (PC Configuration)

FIG. 2 shows a functional configuration of PC (personal computer) that is used in an embodiment of this invention. CPU of the PC processes the data received from mobile electronic devices 1.2.3 through the communication section and outputs the results to the display.

FIG. 3 shows a functional configuration of the mobile electronic device, which is equipped with input unit to input the results of medical interview by SF36 and RDQ, exercise biometric data measurement and collection unit to measure and collect the patient's exercise biometric data, and a communication unit to transmit data of a patient to PC.

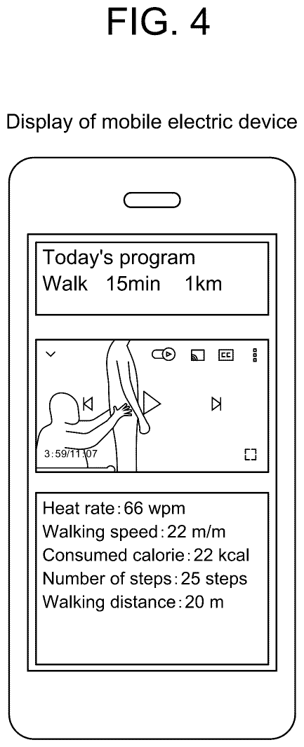

FIG. 4 shows an example of a display screen of a mobile electronic device that is used in an embodiment of the invention.

FIG. 5 shows a sample of SF-36 questionnaire that is used in an embodiment of the invention. FIG. 6 shows a sample of the RDQ questionnaire that is used in an embodiment of the invention. FIG. 7 shows a sample of the questionnaire items of the medical questionnaire that is used in an embodiment of the invention.

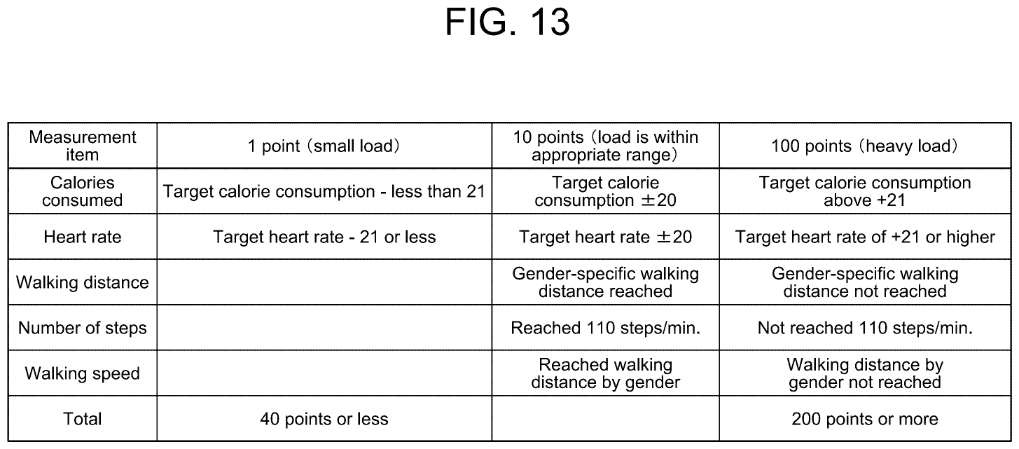

FIG. 13 shows an example of a method of determining overload/underload of an exercise program of a rehabilitation program in an embodiment of present invention. In the embodiment of FIG. 13, calorie consumption, heart rate, walking distance, number of steps, and walking speed of a patient who has performed an exercise of a rehabilitation program are measured.

In FIG. 13, if the measured heart rate is below −20 from the exercise target value of heart rate, 1 point is awarded. If the measured heart rate exceeds +20 from the exercise target value of heart rate of the patient, 100 points are awarded. If the measured heart rate is within ±20 from the exercise target value of heart rate of the patient, 10 points are awarded. Similarly, if the measured calorie consumption is below −20 from the exercise target value of calorie consumption, 1 point is awarded. If the measured calorie. consumption exceeds +20 from exercise target value of calorie consumption, 100 points are awarded. If the measured calorie consumption is within ±20 from the exercise target value of calorie consumption. 10 points are awarded. Similar calculations are made for distance walked, number of steps, and walking speed.

If the total of the points that are awarded to the patient in accordance with above is within a threshold value (ex. 40 points), the exercise program of the prescribed rehabilitation program is judged to be underload for the patient. If the total of the points awarded to the patient above is beyond a threshold value (ex. 200 points), the exercise program of the prescribed rehabilitation program is judged to be overload for the patient.

In another embodiment, if any one of the measurement data of the plurality of measurement items obtained by the mobile electronic device is above/below the threshold value from the exercise target value of the patient, the load of the selected grade of rehabilitation program is determined to have been overload/underload for the patient. In the example shown in FIG. 13, if any one of heart rate, calorie consumption, walking distance, walking steps and walking speed is below or beyond a threshold value from exercise target value, the exercise performed by the patient was underload or overload for the patient.

### Example 1

Referring to FIGS. 8-10, 65-year-old male visited a clinic due to low back pain. At the time of the outpatient visit, he was 170 cm tall, weighed 85 kg, SF-36 score PCS 48, RDQ score 38, both were summed and divided by 2 to obtain an evaluation score of 43, and was determined to have a rehabilitation index of 5 based on the heath evaluation score. As a result, Nordic walking, stretching of the lower back, stretching of the back of the lower extremities, exercises for muscle strength and balance, exercises to strengthen core muscles, back stretching, exercises to improve balance, back extension, and exercises to improve balance were selected as the rehabilitation program (15 minutes per unit) (see FIG. 8). Based on the height (standard) and weight (excess) of the patient male, a target heart rate of 150/min and a target calorie consumption of 148 kcal were set (see FIG. 9(A)). The patient's heart rate at the end of the rehabilitation exercise measured by the mobile electronic device was 160/min and calorie consumption was 160 kcal. Since both the measured patient heart rate of 160/min and calorie consumption of 160 kcal were within the predetermined upper and lower range from the target heart rate (150±10%/min) and target calorie consumption (148±10% kcal), it was decided to maintain grade 5 of the rehabilitation program (see FIG. 10).

### Example 2

Also referring to FIGS. 8-10, a 60-year-old woman visited a clinic due to low back pain. At the time of the outpatient visit, she was 150 cm tall, weighed 60 kg, SF-36 score PCS 26, RDQ score 24, both were summed and divided by 2, resulting in an evaluation score of 25, which was determined to be a rehabilitation index of 3 based on the health evaluation score (FIG. 10). As a result, a rehabilitation program that includes Nordic walking, stretching of the lower back, stretching of the back of the lower limbs, exercises for muscle strength and balance, exercises to strengthen core muscles, back stretching, exercises to improve balance (15 minutes per unit) was selected (see FIG. 8).

Based on the height (standard) and weight (excess) of the patient women, the target heart rate of the selected rehabilitation program was set to be 155/min and the target calorie consumption to be 148 kcal (see FIG. 9(B)).

The heart rate at the end of the patient's rehabilitation exercise as measured by the mobile electronic device was 165/min and calorie consumption was 110 kcal. Since the heart rate (165/min) was within the predetermined upper range from the target heart rate (160±10%/min) and the calories consumed (110 kcal) was below the predetermined lower range from the target calorie consumption (148-10% kcal), the prescribed rehabilitation program was determined to be underload for the patient. Therefore, a rehabilitation program of one level higher grade (rehabilitation index 4) program is proposed (see FIG. 10).

### Example 3

Also referring to FIGS. 8-10, 45-year-old male visited a clinic due to low back pain. At the time of the outpatient visit, he was 160 cm tall, weighed 75 kg, SF-36 score PCS 60 RDQ score 62, both scores were summed and divided by 2, resulting in an evaluation score of 61, which was determined to be a rehabilitation index of 7 based on the results of a medical interview (FIG. 10). As a result, a rehabilitation program that includes Nordic walking, stretching of the lower back, stretching of the back of the lower limbs, exercises for muscle strength and balance, exercises to strengthen core muscles, back stretching, exercises to improve balance (15 minutes per unit) was selected (see FIG. 8). Based on the height (standard) and weight (excess) of the patient male, the target heart rate of the selected rehabilitation program was set to be 140/min and the target calorie consumption to be 132 kcal (see FIG. 9). The heart rate and calorie consumption at the end of the patient's rehabilitation exercise, as measured by the mobile electronic device, were 150/min and 150 kcal, respectively.

Although the patient's measured heart rate of 150/min was within the predetermined range from the target heart rate, the calorie expenditure of 150 kcal was above the upper range from the target calorie consumption. Therefore, it was decided to propose the rehabilitation program of one level lower grade (rehabilitation index 6) (see FIG. 10).

### Example 4

60-year-old female visited a clinic due to osteoarthritis of the knee. At the time of the outpatient visit, she was 156 cm tall, weighed 52 kg, and had an SF-36 score PCS 43 WOMAC score 46, both of which were summed and divided by 2 to obtain an evaluation score of 44.5. Based on the results of the medical interview, the patient was determined to have a rehabilitation index of 5. As a result, a program of 30 standing exercises from a chair (3 sets of 10) for 10 minutes each 3 times a week was selected. Based on the patient woman's height (standard) and weight (excess), the target heart rate was set to be 155/min and the target calorie consumption to be 148 kcal. The patient's heart rate at the end of the rehabilitation exercise, as measured by the mobile electronic device, was 160/minute and calorie consumption was 120 kcal. Since the patient's calorie expenditure was below the predetermined range from the target range set by the rehabilitation program, it was decided to propose the rehabilitation program of one level lower grade.

Although the invention has been explained with reference to the embodiments and drawings, the invention is not limited to these embodiments, but can be modified and implemented in various ways within the scope of the claims.

### DESCRIPTION OF CODE

10 PC (personal computer)
21 Mobile Electronic Devices 1
22 Mobile Electronic Devices 2
23 Mobile Electronic Devices 3

1. A telemedicine support system that assists selecting and executing a rehabilitation program for an orthopedic patient who is capable of performing an independent exercise,
the system comprises a mobile electronic device and a PC (personal computer) that is wirelessly connected to the mobile electronic device,
the mobile electronic device is configured to be carried by a patient, and comprises:
means for accepting an input of identification number, profile, and a kind of disease of the patient,
means for accepting an input of a health evaluation score that quantitatively assesses the patient's health status,
means for measuring and collecting exercise biometric data of the patient, and
means for transmitting the data of the patient to the PC,
the PC is installed in a medical institution and accessible by a physician of the patient, and comprises:
means for receiving data of the patient transmitted from the mobile electronic device over an Internet line, and
a memory device,
the memory device stores a plurality of rehabilitation programs, each of the plurality of rehabilitation programs consists of a plurality of grades in accordance with magnitude of load that is applied to the patient by performing the exercise of the program, each grade of the rehabilitation program is provided with a rehabilitation index of the patient having a health status suitable for the load of the exercise program of the grade,
the memory device further stores a target value of exercise biometric data (exercise target value) for each grade of the rehabilitation program and a profile of the patient who is expected to perform the exercise of the rehabilitation program with the exercise target value,
upon receiving data of the patient from the mobile electronic device, the PC
determines the rehabilitation index that assesses health status of the patient by a numerical scale based on the health evaluation score of the patient,
select a rehabilitation program and a grade of the rehabilitation program to be prescribed for the patient from a plurality of rehabilitation programs stored in the memory based on the kind of disease and the rehabilitation index of the patient, and
determines an exercise target value of the patient for the selected grade of the rehabilitation program based on the profile of the patient,
the system compares a value of exercise biometric data measured from the patient who performed the selected grade of rehabilitation program with the exercise target value of the patient for the selected grade of rehabilitation program,
when the value of the exercise biometric data measured by the mobile electronic device is within a predetermined upper and lower range from the exercise target value, propose using the prescribed rehabilitation program based on a judgement that the rehabilitation program that was prescribed for the patient was not overload nor underload for the patient,
when the value of the exercise biometric data measured by the mobile electronic device is beyond a predetermined upper range from the exercise target value, propose changing the prescribed rehabilitation program into a grade of the rehabilitation program having one level lower load based on a judgement that the rehabilitation program that was prescribed for the patient was overload for the patient, and
when the value of the exercise biometric data measured by the mobile electronic device is below a predetermined lower range from the exercise target value, propose changing the prescribed rehabilitation program into a grade of the rehabilitation program having one level higher load based on a judgement that the rehabilitation program that was prescribed for the patient was underload for the patient.

2. The telemedicine support system of claim 1, wherein the exercise biometric data of the patient is measured over a plurality of measurement items and compared with exercise target value of the patient in accordance with a predetermined rule to determine whether exercise of the prescribed rehabilitation program is overload or underload for the patient.

3. The telemedicine support system of claim 1, wherein the exercise biometric data of the patient comprises data of calorie consumption and heart rate.

4. The telemedicine support system of claim 3, wherein calories consumed by the patient by the exercise of the rehabilitation program is calculated based on the exercise intensity (METs value) of independent exercise and exercise time and patient weight as dictated by the exercise program of the rehabilitation program prescribed to the patient.

5. The telemedicine support system of claim 1, wherein the patient profile includes all or part of the patient's age, gender, height, and weight.

6. The telemedicine support system of claim 1, wherein the exercise biometric data includes all or part of the patient's measured calories consumed, heart rate, walking distance, walking speed, and number of steps.

7. The telemedicine support system of claim 1, wherein the mobile electronic device comprises a mobile phone and a wearable sensor device that can be connected wirelessly or wired to the mobile phone and transmit data of heart rate of the patient to the mobile phone.

8. The telemedicine support system of claim 1, wherein the means of measuring and collecting exercise biometric data of the patient of the mobile electronic device is Google Fit (registered trademark).
